(19) United States
(12) Patent Application Publication
(10) Pub. No.: US 2008/0207847 A1
(43) Pub. Date: Aug. 28, 2008

(54) Furanic-modified amine-based curatives

(76) Inventors: Herman P. Benecke, Columbus, OH (US); Alex W. Kawczak, Dublin, OH (US); Daniel B. Garbark, Blacklick, OH (US)

Correspondence Address: BATTELLE MEMORIAL INSTITUTE, 505 KING AVENUE, COLUMBUS, OH, 43201-2693, US

(21) Appl. No.: 11/903,251

(22) Filed: Sep. 20, 2007

Related U.S. Application Data
(60) Provisional application No. 60/846,259, filed on Sep. 20, 2006.

(51) Int. Cl. C08G 18/00; C07D 307/34

(52) U.S. Cl. 525/454; 549/487; 525/452

(57) ABSTRACT

Difunctional aromatic diamines (e.g. Ethacure® 100 and 300) are derivatized with furan-2,5-dicarboxylic acid (FDCA) to form FDCA-derived bisamides; the derivatives have enhanced curative properties when used as curatives for polyureas, hybrid epoxy-urethanes, hybrid urea-urethanes, chain extenders for polyurethane and polyurea elastomers, and also for reaction injection molding (RIM) products.

This application claims the benefits of U.S. Provisional Application 60/846,259, filed Sep. 20, 2006.

### FIELD OF THE INVENTION

In a typical embodiment difunctional aromatic diamines (e.g. Ethacure® 100 and 300) are derivatized with furan-2,5-dicarboxylic acid (FDCA) to form FDCA-derived bisamides. The derivatives have enhanced curative properties when used as curatives for polyureas, hybrid epoxy-urethanes, hybrid urea-urethanes, chain extenders for polyurethane and polyurea elastomers, and also for reaction injection molding (RIM) products. The products of the present invention allow control of cure rate for improved performance with complex molds.

### BACKGROUND OF THE INVENTION

Difunctional or polyfunctional aromatic amines currently have a variety of uses as curatives in reacting with polyisocyanates or mixtures of polyisocyanates and alcohols to form urea and urea/urethane derivatives, respectively. Ethacure® 100 is a commercially available aromatic diamine curative derived from toluene that is further substituted with two ethyl groups on the aromatic ring. Ethacure® 300 is another commercially available aromatic diamine curative derived from toluene that is further substituted with two methylthio groups. The following Formulas A1 and A2 show the major and minor components, respectively, in Ethacure® 100 and Formulas A3 and A4 show the major and minor components, respectively, in Ethacure® 300:

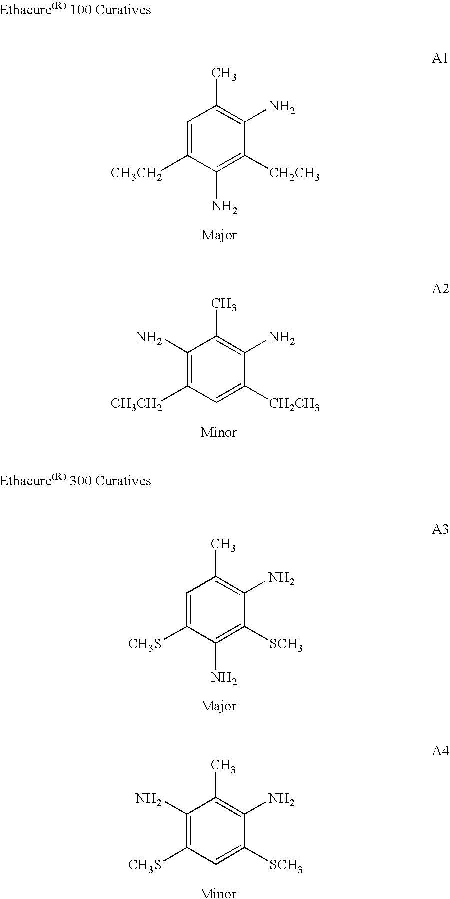

The major and minor constituents are in a ratio of about 80 major to 20 minor for both curatives.

Ethacure® 100 is used as a curative agent for polyurethanes and polyureas, and a chain extender for polyurethane and polyurea elastomers, and particularly in reaction injection molding (RIM) and spray applications. Ethacure® 300 has shown special utility when used with MDI and TDI polyether and polyester prepolymers. The relative reactivity of these two curatives with isocyanates is such that Ethacure® 100 reacts appreciably faster than Ethacure® 300.

A general problem with these and other aromatic diamine or polyamine curatives is that they have limited “pot lives” (gel times) because their high reaction rates with polyisocyanates, mixtures of polyisocyanates and alcohols, and epoxides cause problems in various applications. These high reaction rates, and the resulting short “pot lives” can result in undesirable surface aesthetics and physical properties in coatings, adhesives, sealants, castings and moldings prepared from these curatives. Another serious liability of these type curatives, due to their limited “pot lives”, for a number of products and applications is that when mixtures of these curatives with polyisocyanates and(or) alcohols, or epoxides are placed in complex molds they tend to set-up prematurely before the mold can be completely filled, resulting in partial and incomplete mold filling. The products obtained from molds having this problem can have hidden or obvious defect structures causing low productivity and inferior products. The present invention prevents or reduces these problems.

### BRIEF DESCRIPTION OF THE INVENTION

Broadly the invention discloses an aromatic amine bisamide of furan-2,5-dicarboxylic acid having the structure (AB)ₙA;

wherein A is an aromatic diamine moiety, B is a furan-2,5-dicarboxylic acid moiety and n is an integer from 1 to 10;
wherein each aromatic diamine moiety in the bisamide comprises 0, 1, 2, 3, 4, or 5 substituents selected from the group consisting of alkyl, aryl, alkylaryl, halogen, nitro, carboxyl, carbonyl, primary amino (—NH₂), secondary amino (—NHR), tertiary amino (—NR₂), aminoalkyl (—RNH₂), hydroxyl (—OH), alkoxy (—OR), hydroxylalkyl (—ROH), thiol (—SH), and alkylthio (—SR), wherein at least one group is either a primary or secondary amino, aminoalkyl, hydroxyl, or thiol group, and the remaining positions are occupied by H; and
wherein each group may contain between 1 to 10 carbon atoms. In some embodiments the group may contain up to 6 carbon atoms. Typically the alkylthio group comprises the methylthio group.

Another embodiment of the invention includes the aromatic amine bisamide of furan-2,5-dicarboxylic acid described above wherein the specific positional labeling of the two nitrogen atoms in the major species A1 and A2 of the Ethacure® 100 series diamines is as follows:

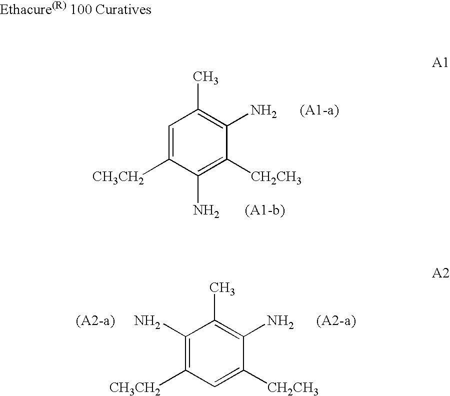

wherein the positional specificity of individual bisamides is specified by the following generically labeled structures where the label AX-y (where X=1 or 2 and y=a or b) specifies the specific aromatic nitrogen atom involved in amide bond formation:

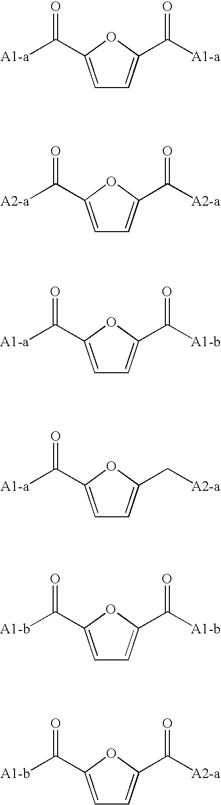

A further embodiment of the invention includes the aromatic amine bisamide of furan-2,5-dicarboxylic acid disclosed above where the specific positional labeling of the two nitrogen atoms in the major species A1 and A2 of the Ethacure® 300 series diamines is as follows:

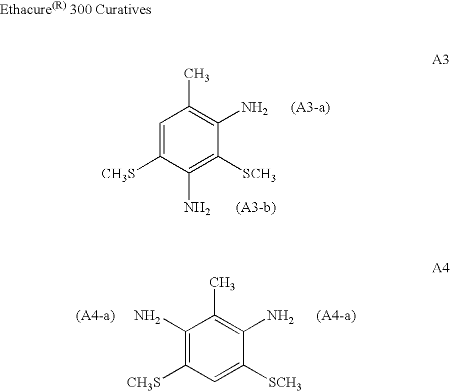

wherein the positional specificity of individual bisamides is specified by the following generically labeled structures where the label AX-y (where X=3 or 4 and y=a or b) specifies the specific aromatic nitrogen atom involved in amide bond formation:

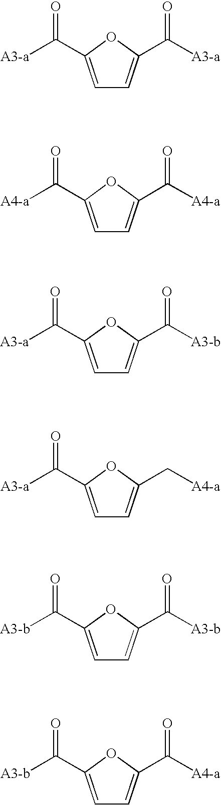

A further broad embodiment includes the composition

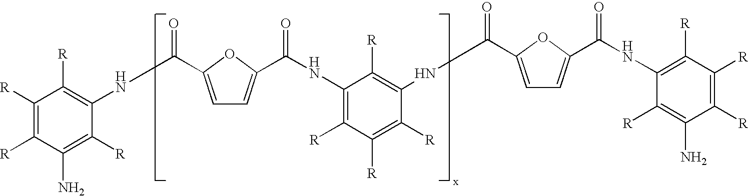

Wherein x=0, have A-B-A structure;
Wherein x=1 have A-B-A-B-A type structure;
Wherein x may have any value from 0 to 9;
the amino (—NH₂) groups on the substituted phenyl ring may be meta, ortho, or para with respect to each other,
R may be the same or different, and is selected from the group consisting of alkyl, aryl, alkylaryl, halogen, nitro, carboxyl, carbonyl, primary amino (—NH₂), secondary amino (—NHR′), tertiary amino (—NR₂′), aminoalkyl (—R′NH₂), hydroxyl (OH), alkoxy (—OR′), hydroxylalkyl (—R′OH), thiol (—SH) and alkylthio (—SR′), wherein the remaining positions are occupied by H, and wherein the R an R′ groups may contain 1 to 10 carbon atoms.

Another embodiment of the invention includes a method for controlling cure time and (or) pot life of polyurea, hybrid epoxy-urethane, and hybrid urea-urethane chain extenders for polyurethane and polyurea elastomer systems by the steps of:

a. using an aromatic diamine curative, wherein the aromatic diamine is replaced to varying amounts with an furan-2,5-dicarboxylic acid bisamide of such aromatic diamine, wherein increasing amounts of furan-2,5-dicarboxylic acid bisamide lead to reduced reaction rates that provide increased pot life and longer reaction time.

A yet further embodiment includes a method for making furan-2,5-dicarboxylic acid bisamide by the steps of:

a. providing a furan-2,5-dicarboxylic acid diacid chloride, an aromatic diamine, an optional catalyst and a solvent;
b. mixing the furan-2,5-dicarboxylic acid diacid chloride with the aromatic diamine in the solvent, optionally in the presence of the catalyst; and
c. reacting the mixture of step b, optionally under heat, until the furan-2,5-dicarboxylic acid bisamide is formed. Typically the product furan-2,5-dicarboxylic acid bisamide is separated from the reaction mixture. The furan-2,5-dicarboxylic acid bisamide containing solvent is typically separated by filtration, the higher oligomers remaining behind.

A yet additional embodiment includes a method for separating a furan-2,5-dicarboxylic acid bisamide having the formula (A-B)ₙA wherein n=1, from higher oligomers having the formula (A-B)ₙA wherein n is greater or equal to 2, comprising the steps of obtaining a mixed (A-B)ₙ-A product, wherein n is 1 to greater than 1; fractionating the mixed product with a solvent in which the A-B-A is more soluble than the higher oligomers, wherein the A-B-A product is dissolved in the solvent. Typically the solvent is moderately polar and exemplified by acetonitrile. The solvent containing A-B-A product is typically removed by from the higher oligomers by filtration.

Another embodiment of the invention includes a method for making a furan-2,5-dicarboxylic acid bisamide comprising:

a. providing furan-2,5-dicarboxylic acid, aromatic diamine, triphenyl phosphite, and pyridine;
b. mixing furan-2,5-dicarboxylic acid, aromatic diamine, triphenyl phosphite, and pyridine; in solvent; and
c. reacting the mixture under optional heating until the furan-2,5-dicarboxylic acid bisamide is formed. Typically the heating produces a temperature of about 80° C. to about 110° C.

A still further embodiment of the invention includes a method for making a furan-2,5-dicarboxylic acid bisamide by the steps of:

a. providing furan-2,5-dicarboxylic acid, aromatic diamine, a molecular sieve Zeolite® and an optional solvent;
b. mixing furan-2,5-dicarboxylic acid, aromatic diamine, molecular sieve (e.g. Zeolite® and with or without the solvent; triphenyl phosphite, and pyridine; in solvent; and
c. reacting the mixture with microwave radiation until the furan-2,5-dicarboxylic acid bisamide is formed.

Another embodiment of the invention includes a method for making a furan-2,5-dicarboxylic acid bisamide comprising:

a. providing furan-2,5-dicarboxylic acid, aromatic diamine, phosphorous pentachloride, and solvent;
b. mixing furan-2,5-dicarboxylic acid, aromatic diamine, phosphorous pentachloride, and solvent and heating; and
c. reacting the mixture until the furan-2,5-dicarboxylic acid bisamide is formed.

### BRIEF DESCRIPTION OF THE DRAWINGS

FIG. 1 is a graph illustrating HPLC analysis of a crude Ethacure® 100 reaction mixture showing components having (AB)ₙA structures where n=1, 2, and 3.

FIG. 2 is a graph illustrating HPLC analysis of an acetonitrile soluble Ethacure® 100 reaction mixture showing components having (AB)ₙA structures where n is primarily 1.

FIG. 3 is a graph illustrating HPLC analysis of an acetonitrile insoluble Ethacure® 100 reaction mixture showing components having (AB)ₙA structures where n is primarily 2, while also showing minor amounts of (AB)ₙA structures were n=1 and 3.

FIG. 4 is a graph illustrating viscosity versus time plots for curing of Ethacure® 100, of FDCA bisamide of Ethacure® 100, and of mixtures thereof.

### DETAILED DESCRIPTION OF THE INVENTION AND BEST MODE

A general solution to reduce or eliminate the problems discussed above is to reduce the reactivity of these type polyamine curatives by preparing derivatives of these curatives in which the reactivity of amine groups have been reduced. The inventive method described herein to reduce the reactivity of aromatic amine curatives is to prepare amide linkages between at least one amino group of such curatives with the carboxylic acid group of an electron deficient polyacid. The use of electron deficient polyacids to form these amide linkages causes the remaining non-acylated amine groups of the aromatic polyamine curatives to have significantly reduced electron density due to electron withdrawing effects. Thus, the non-acylated amine groups are expected to have decreased nucleophilicities and reactivities towards isocyanates. These deactivated polyamine-based curatives are also expected to have decreased reactivities towards carboxylic acids or carboxylic acid derivatives such as acid anhydrides and acid halides. These deactivated amine based curatives can then be used solely or in formulated blends containing Ethacure® 100, Ethacure® 300, 4,4′-methylene bis(2-chloroaniline) (MOCA), and other commercially available amine curatives by improving the processing and performance of polyurethane, polyurea, epoxy, and hybrid (urethane-epoxy, urethane-phenolic) adhesives, coatings, foundry binders, elastomers, composites and sealants. Typically, other aromatic amine curatives can be used.

An electron deficient polyacid that fits these requirements is furan-2,5-dicarboxylic acid (FDCA) that has an unusually low pKa(1) value of 2.60 indicating that it is a significantly stronger acid than benzoic acid (pKa=4.20) or acetic acid (pKa=4.75). The pKa value of an acid is the negative of the logarithm of its acidity constant (Ka) so that the lower the pKa, the higher the acidity of the acid. Higher acidities are caused by more effective electron withdrawal from and stabilization of the carboxylate group (the conjugate base) derived from the carboxylic acid group. Another advantage of FDCA is that it is biobased and thus sustainable since it is derived from 5-hydroxylmethylfurfural (HMF) which is derived from the cyclic dehydration of fructose or other six carbon monosaccharide ketoses or aldoses. Another advantage of incorporating FDCA in these curatives is that they should benefit from the flame and smoke inhibiting properties inherent in furanic compounds.

The bisamide of FDCA prepared from the major isomer of Ethacure® 100 is shown below (only one of the three possible regioisomeric forms incorporating the major isomer is shown):

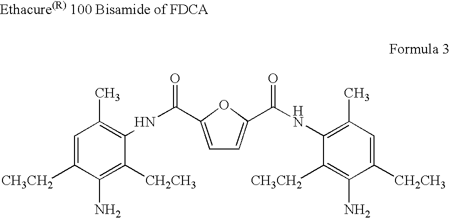

This type FDCA bisamide has two available amino groups that are expected to have reduced nucleophilic reactivity in reaction with isocyanate functionality to produce urethane linkages and with carboxylic acids (or acid anhydrides or acid halides) to produce additional amide linkages due to the reduced electron densities of the non-derivatized amine nitrogen atoms. These type products are also expected to be relatively rigid due to the known hindered rotation within amide groups that would be coupled with the planar structure of FDCA to give products derived from these modified curatives with potentially higher tensile strengths and toughness. These type derivatives also have two amido hydrogen atoms that can enter into allophanate formation and thus serve as tetra-functional crosslinking components.

The above class of FDCA bisamides of Ethacure® 100 can be referred to as A-B-A derivatives where A is an Ethacure® 100 moiety and B is an FDCA moiety. This product has been prepared by a number of approaches including:

(1) reaction of FDCA diacid chloride with Ethacure® 100,
(2) reaction of FDCA and Ethacure® 100 in the presence of phosphorous pentachloride (that produces FDCA acid chloride in-situ),
(3) reaction of FDCA with Ethacure® 100 using triphenyl phosphine and pyridine as co-reagents,
(4) reaction of FDCA with Ethacure® 100 in the presence of Zeolite H-Y® using microwave radiation as the energy source, and
(5) reaction of FDCA dimethyl ester with Ethacure® 100 using sodium iodide or sodium methoxide as catalysts.

Regardless of the method of preparation, an excess of Ethacure® 100 typically and preferably is used to promote primary production of A-B-A derivatives and minimize the production of (AB)ₙA oligomers, where n>1. Even when an excess of Ethacure® 100 was used in these approaches, some oligomeric polyamides of the formula (AB)ₙA were formed where n=2 (structure A-B-A-B-A), n=3 (structure A-B-A-B-A-B-A), and possibly n>3. One of the oligomers having the A-B-A-B-A structure (where the major component of Ethacure® 100 is used) is shown in Formula 4, below:

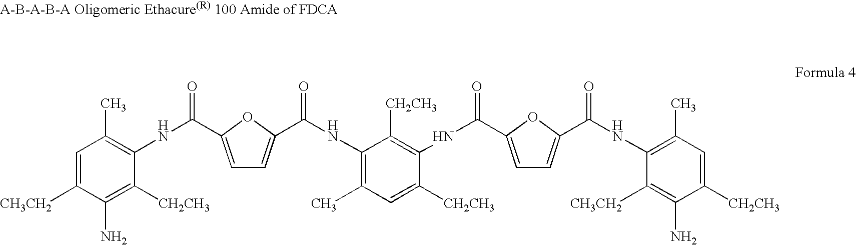

A further embodiment of the invention is illustrated by Formula 5, below. Formula 5 represents the general case for a structure (AB)ₙA where various substituents are represented by the —R group.

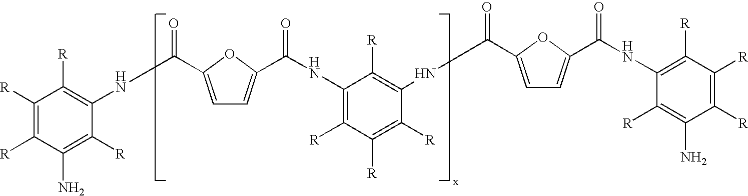

When x=0, have A-B-A structure.
When x=1 have A-B-A-B-A type structure.

Wherein x may have any value from 0 to 9.

The original —NH₂ groups on the substituted phenyl ring may be meta, ortho, or para with respect to each other
R may be the same or different, and is typically selected from the group consisting of alkyl, aryl, alkylaryl, halogen, nitro, carboxyl, carbonyl, primary amino (—NH₂), secondary amino (—NHR′), tertiary amino (—NR₂′), aminoalkyl (—R′NH₂), hydroxyl (OH), alkoxy (—OR′), hydroxylalkyl (—R′OH), thiol (—SH) and alkylthio (—SR′), wherein the remaining positions are occupied by H. wherein the R an R′ groups may contain 1 to 10 carbon atoms.

### Formula 5 (Above)

A further embodiment is found in Formula 6, where given the specific positional labeling of the two nitrogen atoms in the major species A1 and A2 of the Ethacure® 100 series diamines as follows:

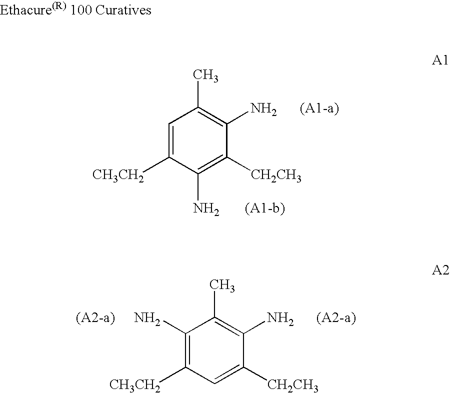

included are the aromatic amine bisamides of furan-2,5-dicarboxylic acid having the structure (A-B)ₙ-A where n=1-10 where A can be A1 or A2, whereby the positional specificity of individual bisamides is specified by the following generically labeled structures where the label AX-y (where X=1 or 2 and y=a or b) specifies the specific aromatic nitrogen atom involved in amide bond formation:

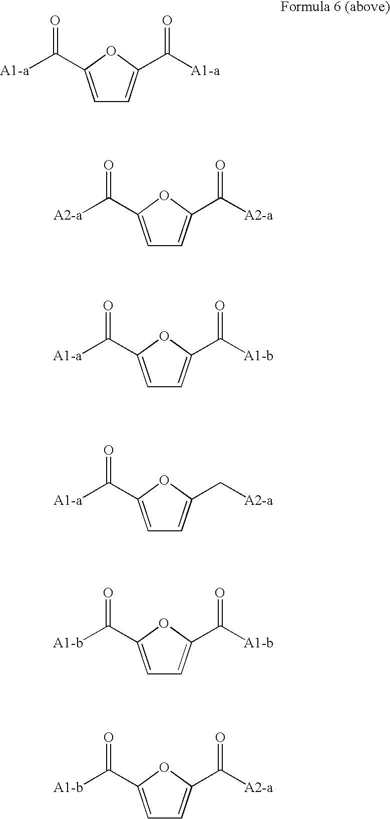

Another typical embodiment shown in Formula 7 below, includes the following: given the specific positional labeling of the two nitrogen atoms in the major species A1 and A2 of the Ethacure® 300 series diamines as shown below:

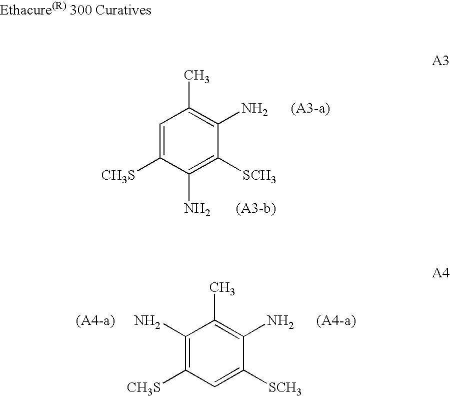

included are the aromatic amine bisamides of furan-2,5-dicarboxylic acid having the structure (A-B)ₙ-A where n=1-10 where A can be A3 or A4, wherein the positional specificity of individual bisamides is specified by the following generically labeled structures where the label AX-y (where X=3 or 4 and y=a or b) specifies the specific aromatic nitrogen atom involved in amide bond formation:

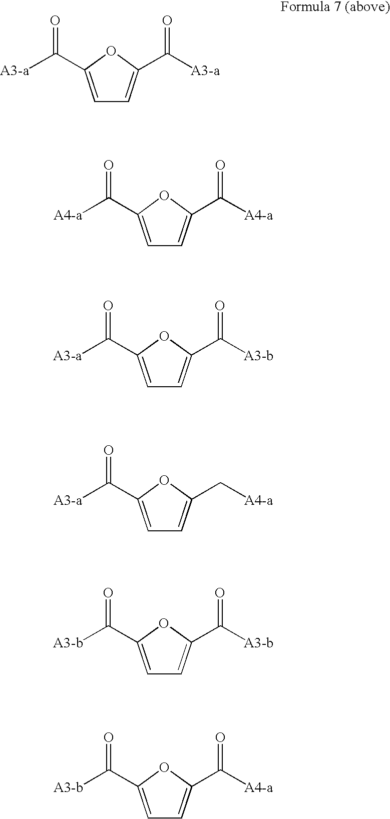

Each of the (AB)ₙA oligomeric polyamides is bifunctional in that each is terminated with an amino group at both ends of these oligomers. These oligomers are also expected to have relatively rigid structures, due to the planarity of the furanic ring and the relatively hindered rotation about the amide carbonyl carbon atom and nitrogen atom, that may beneficially contribute to the tensile strengths and toughness of products derived from these modified curatives.

A convenient method for separating FDCA bisamides of Ethacure® 100 (A-B-A systems) from higher (AB)ₙA oligomers has been discovered that involves use of solvents that have relatively high solubility for A-B-A bisamides and relatively low solubility for higher (AB)ₙA oligomers where n≧2. Typically these solvents include, without limitation, moderately polar solvents such as acetonitrile. Thus, simply stirring a mixture of an FDCA Ethacure® 100 amide product mixture with acetonitrile will result in relatively efficient solubilization of FDCA bisamides of the formula (AB)ₙA, where n=1. The FDCA bisamides of Ethacure® 100 where n=1 can be isolated in relatively high purity by filtering the acetonitrile solution and evaporating the filtrate to dryness, whereas the insoluble material filtered from acetonitrile is rich in oligomeric polyamides of the formula (AB)ₙA, where n≧2. These two fractions of amine curatives should give rise to products having different curative properties due to the different molecular sizes and shapes of these derivatives.

Data disclosed herein indicates that the available non-reacted amino groups of A-B-A Ethacure®100 moieties in FDCA bisamides have significantly reduced reactivity towards polyisocyanates compared to those of non-derivatized Ethacure® 100.

FDCA bisamides derived from Ethacure® 300 as well as from other aromatic diamines will have similar structures as those shown above for Ethacure® 100.

### EXAMPLES

The following examples illustrate the preparation, fractionation and characterization of FDCA bisamides of Ethacure® 100 and higher oligomeric amides by reaction of Ethacure® 100. The examples are illustrative only and are not intended to limit the scope of the invention in any way.

### Example 1

This example illustrates the reaction of an aromatic diamine (Ethacure® 100) with an FDCA diacid chloride to produce amides having (AB)ₙA structures.

FDCA diacid chloride was prepared from FDCA and phosphorous pentachloride as described in the chemical literature {J. Lewkowski, Polish J. Chem., 75, 1943-1946 (2001)}. FDCA diacid chloride (55.10 g; 0.286 mole) was weighed into a pressure-equalizing addition funnel and dissolved in 300 mL diethyl ether under an argon blanket. Ethacure® 100 (408.21 g; 2.29 mole) and triethylamine 64.07 g; 0.633 mole) were weighed into a three-neck round bottom flask containing a magnetic stir bar, a thermocouple, and an argon gas inlet tube and the reaction flask was positioned in a heating mantle. Toluene (1000 mL) and hexane (350 mL) was added to the reaction vessel and this mixture was flushed with argon while maintaining the reaction flask under positive argon pressure by delivering argon through a bubbler filled with mineral oil. The addition funnel containing FDCA diacid chloride was then attached and this solution was then added drop-wise over two hours into the stirred Ethacure® 100 solution (magnetic stir bar) while reaction mixture temperature increased to 30.5° C. The addition funnel was rinsed with 50 mL toluene and the contents were added to the reaction flask. The reaction mixture was then heated to 45° C. for one hour with mechanical stirring after which the solution was cooled to room temperature. The mixture was then filtered through a coarse fritted Buchner funnel and rinsed with two 200 mL portions of 50% toluene in hexane and one 200 mL portion of hexane. The remaining solid material was dried in a vacuum oven containing phosphorus pentoxide for about 3 hours. Once dry, the material was ground into a powder (233.26 g) and NMR spectroscopy indicated a significant amount of residual triethylamine hydrochloride. The powder was then placed into an Erlenmeyer flask containing water (1 L) and a stir bar. The mixture was heated to 50° C. and stirred for five (5) hours. The resulting solid was filtered, rinsed with four 500 mL portions of water, and dried in a vacuum oven with phosphorus pentoxide. NMR analysis indicated that this material contained approximately 14.9 percent triethylamine hydrochloride on a mole basis. Water washing was repeated at 50° C. for four hours (4) and the solid was dried in a vacuum oven with phosphorus pentoxide. NMR spectroscopy of this material revealed that the triethylamine hydrochloride had been reduced to 8.3 percent on a mole basis. HPLC analysis supported the desired product. Water washing was then performed at 80° C. for 1.5 hours and the mixture was filtered while hot. The solid was placed into a vacuum oven with phosphorus pentoxide overnight. NMR spectroscopy revealed a negligible quantity of triethylamine hydrochloride.

HPLC analysis of crude product on a reverse phase column using acetonitrile/water (57:43) at a flow rate of 1 ml/min was consistent with the presence of amides having (AB)ₙA structures where n=1, 2, and 3 and indicated that the major component had an A-B-A structure and minor components had n values of 2 and 3 (see FIG. 1). Given the regioisomeric bonding potential in the two positional isomers in Ethacure® 100, it can be seen that the A-B-A bisamide can theoretically exist in up to six isomeric forms, three A-B-A bisamide isomers would be derived exclusively from the major Ethacure® 100 isomer, one A-B-A bisamide isomer would be derived from the minor Ethacure® 100 isomer (realizing that this minor isomer has a plane of symmetry so each amino group is equivalent), and two A-B-A bisamide isomers would be derived from cross reaction of the major and minor Ethacure® 100 isomers. Thus, the five closely separated peaks in the 6-9 minutes retention time region in this chromatogram were assigned to five of these six possible isomers of the (AB)ₙA where n=1 (FDCA bisamide of Ethacure® 100). The potential number of possible structural isomers increases significantly in higher (AB)ₙA oligomers where n≧2. It can be seen that another cluster of peaks (with significantly decreased peak intensities) lies in the 9 to 14 minute retention time region and this cluster is assigned to (AB)ₙA oligomers where n=2. Another cluster of peaks (with still lower peak intensities) with retention times greater than 14 minutes are presumed to correspond to (AB)ₙA oligomers where n=3. Based on the relative integration of peaks in the A-B-A region versus higher retention time regions, it was estimated that the A-B-A components comprised approximately 74.4% of this mixture.

### Example 2

This example illustrates the isolation and structural characterization of a predominantly A-B-A Product.

To separate the A-B-A product from higher oligomers, the isolated solid from Example 1 was dissolved into 3000 mL acetonitrile and the mixture was stirred approximately 15 minutes. The mixture was then filtered through a coarse fritted Buchner funnel containing Celite® and the filtrate was placed on a rotary evaporator and acetonitrile was removed by aspirator vacuum. The sample was dried completely in a vacuum oven with a vacuum pump and the resulting green solid (predominately A-B-A) weighed 65.16 g (47.9% yield). Note that the Celite® (diatomaceous earth) is used as a filtration aid and is optional in the process.

HPLC analysis of acetonitrile-soluble product indicated that the percentage of A-B-A product (FDCA bisamide of Ethacure® 100) was approximately 89.6% of the total product mixture (see FIG. 2).

Proton NMR Spectroscopy. The 500 MHz proton NMR spectrum (in DMSO-d6) of the acetonitrile fractionated product described above supported the desired structure based on the presence of amide protons at 9.7-10.05 ppm, furanic protons at 7.25-7.41 ppm, phenyl protons at 6.4-7.2 ppm, amine protons at 4.2-4.8 ppm, methyl protons at 1.8-2.25 ppm., and protons from the methyl groups of the phenyl ethyl groups at 0.95-1.20 ppm. The methylene groups of these ethyl groups overlap significantly with the solvent peak in the same region. The relative integration values of these regions are close to those expected for the A-B-A structure.

Infrared Spectroscopy. The infrared spectrum of this product shows a prominent peak at 1648 cm⁻¹ which is consistent with aromatic amides.

Matrix assisted laser desorption ionization time of flight mass spectroscopy (MALDI TOF MS) was performed on a similarly obtained acetonitrile soluble fraction and the results were consistent with the predominance of FDCA bisamides of Ethacure® 100. Table 1 shows the relative peak areas of (AB)ₙAH+ species when 2,5-dihydroxybenzoic acid (DHB) and trans-3-indoleacrylic acid (IM) were used to generate protonated species. Table 2 shows the relative peak areas of (AB)ₙANa⁺species generated by the natural presence of sodium ions that were also observed when DHB and IAA were used as ionizing species. It can be seen that integration of peaks corresponding to protonated species indicates a higher percentages of lower molecular weight (AB)ₙA species than integration of sodium ion-complexes.

Tables 1 and 2. MALDI TOF MS Analysis of Acetonitrile-Soluble Ethacure® 100 Reaction Mixture

TABLE 1

| | | Relative | Relative |
|---|---|---|---|
| | m/z | Area using | Area using |
| [AB]ₙAH⁺ | (MH⁺) | DHB | IAA |
| n = 1 | 477.4 | 91% | 95% |
| n = 2 | 775.4 | 6% | 2% |
| n = 3 | 1073.6 | 3% | 3% |
| n = 4 | 1371.7 | 0% | — |

TABLE 2

| | | Relative | Relative |
|---|---|---|---|
| | m/z | Area using | Area using |
| [AB]ₙANa⁺ | (MNa⁺) | DHB | IAA |
| n = 1 | 499.4 | 70% | 68% |
| n = 2 | 797.5 | 15% | 14% |
| n = 3 | 1095.7 | 14% | 15% |
| n = 4 | 1393.8 | 1% | 2% |

Titration of a similarly obtained acetonitrile soluble fraction dissolved in chlorobenzene with 0.1703 N perchloric acid in glacial acetic acid using methyl violet as an indicator indicated that the amine content was 97.3% of that expected for the A-B-A structure and indicated that the amino group concentration was 4.08 mmole amino group per gram of sample. The theoretical value of an A-B-A FDCA bisamide of Ethacure® 100 is 4.20 mmole amino groups per gram. These differences appeared to be due to the presence of small amounts of (AB)ₙA species where n≧2. Titration of Ethacure® 100 itself indicated the amine concentration was 95.8% of theoretical, which validates this titration in the Ethacure® 100 system.

### Example 3

This example illustrates the isolation and structural characterization of the acetonitrile-insoluble product from above -(AB)ₙA where n is predominantly 2 and 3.

The acetonitrile-insoluble product obtained from the acetonitrile extraction described above was extracted with acetone (1.0 L), tetrahydrofuran (1.2 L), and isopropanol (500 mL) to remove the oligomeric amide product from (Celite®) used in the prior filtration. After stripping on a rotary evaporator and further drying in a vacuum oven with vacuum pump pressure, 17.1 g of a light brown powder was obtained. This weight corresponds to 12.6% additional yield.

HPLC Analysis. HPLC analysis of the acetonitrile-insoluble product indicated that this mixture is composed of approximately 94.1% of (AB)ₙA product where n≧2 and 5.9% where n=1 (see FIG. 3).

Proton NMR Spectroscopy. The 500 MHz proton NMR spectrum (in DMSO-d6) of a similarly obtained non-acetonitrile soluble fractionated product described above supports the similarity to desired structure based on the presence of amide protons at 9.7-10.05 ppm, furanic protons at 7.25-7.41 ppm, phenyl protons at 6.4-7.2 ppm, amine protons at 4.2-4.8 ppm, methyl protons at 1.8-2.25 ppm., and protons from the methyl groups of the phenyl ethyl groups at 0.95-1.20 ppm. The methylene groups of these ethyl groups overlap significantly with the solvent peak in the same region. The relative integration values of these regions are close to those expected for the A-B-A-B-A structure.

### Example 4

This example illustrates the reaction of aromatic diamine with FDCA diacid chloride prepared in-situ.

FDCA (5.00 g; 0.032 mole) was weighed into a round bottom flask with Ethacure® 100 (22.92 grams; 0.129 mole) under an argon blanket. Toluene (75 mL) and hexane (75 mL) were added to the reaction flask and the mixture was stirred under argon. Phosphorus pentachloride (1.62 g; 0.008 mole) was weighed under argon and added to the reaction flask with stirring. A magnetic stir bar, a contact thermocouple, a heating mantle, and a condenser with an argon gas inlet tube were added to the reaction flask. The mixture was then refluxed for four (4) hours after which an IR spectrum revealed that the FDCA peak at 1680 cm⁻¹ was no longer present but there was a shoulder on the left side of the peak at 1625 cm⁻¹. The mixture was further refluxed for 3 hours and the resulting mixture was filtered. The solid was washed with two 100 mL portions of 50% toluene in hexane followed by two 100 mL portions of hexane. The residual solvent was removed by vacuum. NMR spectroscopy was run on the resulting 19.2 grams of material to reveal 37.8% by mole product with the balance being FDCA starting material. This corresponds to 65% by weight product and 35% by weight FDCA. This conversion could be improved by increased reflux time. Based on component solubilities, these mixtures can purified by dissolving in acetonitrile and filtering the unreacted FDCA to allow isolation of purified product by stripping the solvent with a rotary evaporator.

### Example 5

This example illustrates the reaction of an aromatic diamine with FDCA using triphenyl phosphite and pyridine as co-reagents.

FDCA (4.99 g; 0.032 mole) was weighed into a three-neck round bottom flask, containing a stir bar, and Ethacure® 100 (23.69 g; 0.133 mole), calcium chloride (9.81 g), lithium chloride (3.35 g) and triphenyl phosphite (23.90 g; 0.076 mole) were added under an argon blanket. Pyridine (33.5 mL) and 1-methyl-2-pyrrolidinone (165 mL) were added to the flask and an argon gas inlet and a heating mantle with a contact thermocouple were attached to the flask. The mixture was heated to 90° C. for 20 hours and the mixture was then poured into water (1600 mL) and stirred for 3 hours. The water was decanted from the oil and the oil was rinsed with 50% toluene in hexane (300 mL). The oil was placed into a vacuum oven and dried with phosphorus pentoxide under vacuum pump pressure. Solid material was scraped from flask to obtain 14.26 g product that corresponds to a 93.7% yield. The product was verified by NMR spectroscopy and HPLC analysis

### Example 6

This example illustrates the reaction of an aromatic diamine with FDCA using triphenyl phosphite and pyridine as co-reagents without solvent (neat).

FDCA (5.00 g; 0.032 mole) was weighed into round bottomed flask containing a magnetic stir bar, Ethacure® 100 (22.94 g; 0.129 mole), triphenyl phosphite (22.00 g; 0.071 mole), and pyridine (6.00 mL; 0.074 mole) under an argon blanket. The mixture was heated to 100° C. for 20 hours. NMR spectroscopy was taken and verified the production of the FDCA bisamide.

The product was purified by extraction with a 50/50 hexane/toluene, water mixture. Product was isolated from the hexane/toluene layer.

### Example 7A

This example illustrates the reaction of an aromatic diamine with FDCA in the presence of Zeolite H-Y® with conventional heating and microwave radiation, and without conversion of FDCA to its acid chloride.

FDCA (5.00 g; 0.032 mole) was weighed into a round bottom flask with Ethacure® 100 (22.82 grams; 0.128 mole) and Zeolite H-Y® (1.05 g) under an argon blanket. The mixture was heated with conventional heating to 210° C. for 4 hours. IR spectroscopy showed no new peaks indicating no product was formed.

The mixture was then subjected to microwave radiation in a 1250 watt microwave oven for 60 sec. NMR spectroscopy revealed a small amount of amide present based on the appearance of a peak at 9.75 ppm. These results showed that, surprisingly, normal heating does not promote this reaction, but energy supplied in the form of microwave radiation does promote this reaction.

### Example 7B

This example illustrates the reaction of an aromatic diamine (Ethacure® 100) with FDCA in the presence of Zeolite H-Y® and with the application of microwave radiation.

FDCA (5.00 g; 0.032 mole) was weighed into an Erlenmeyer flask with Ethacure® 100 (22.83 g; 0.128 mole) and Zeolite H-Y® (1.00 g) under an argon blanket. The mixture was warmed slightly above room temperature to allow effective mixing. The mixture was then subjected to microwave radiation in a 1250 watt microwave for five (5) minutes followed by a four (4) minute microwave treatment. The two stage heating process was used to prevent overheating of the reactants.

NMR spectroscopy verified a 35.3 percent by mole production of the FDCA bisamide product.

This example confirmed the unexpected and surprising result that the application of microwave radiation resulted in a good yield of FDCA bisamide product.

### Example 8

This example illustrates the preparation and characterization of FDCA bisamides of Ethacure® 300 and higher oligomeric amides by reaction of an aromatic diamine (Ethacure® 300) with FDCA diacid chloride.

FDCA diacid chloride was prepared from FDCA and phosphorous pentachloride as described in the chemical literature {J. Lewkowski, Polish J. Chem., 75, 1943-1946 (2001)}. FDCA diacid chloride (27.14 g; 0.141 mole, 74.0% pure) were weighed into a equal pressure addition funnel and dissolved in 150 mL toluene and 100 mL diethyl ether under an argon blanket. Ethacure® 300 (177.60 g; 0.830 mole) and triethylamine (43.10 g; 0.426 mole) were weighed into a three-neck round bottom flask containing a magnetic stir bar, thermocouple, and argon gas inlet tube. Toluene (400 mL) was added and this mixture was flushed with argon and the system was maintained under positive argon pressure by delivering argon through a bubbler filled with mineral oil. The addition funnel containing the FDCA diacid chloride was then attached to the solution and added drop-wise over one hour to the rapidly stirred solution while allowing the temperature to rise to 43° C. The addition funnel was rinsed with 50 mL toluene and a heating mantle was attached to flask. The mixture was then maintained at 45° C. for one hour. After cooling to room temperature, the mixture was then filtered through a coarse fritted Buchner funnel and rinsed with two 150 mL portions of toluene, two 150 mL portions of hexane, and two 200 mL portions of water (to dissolve the bulk of the triethylamine hydrochloride byproduct). The solid was then stirred 19 hours in 400 mL water at ambient temperature, filtered, rinsed with two 150 mL portions of water, and then dried in a vacuum oven with phosphorus pentoxide under vacuum pump pressure. Proton NMR spectroscopy showed the continued presence of triethylamine hydrochloride so the solid was then washed with 500 mL water while stirring at 55-60° C. for 18 hours. After cooling the mixture to ambient temperature, the solid was filtered and rinsed with three 200 mL portions of water. The solid was placed into a vacuum oven containing phosphorus pentoxide to remove water using vacuum pump pressure to obtain a tan solid weighing 26.20 g (45.9% Yield). The product was found to be mainly insoluble in acetonitrile so this material was not fractionated into FDCA bisamides of Ethacure® 300 (A-B-A systems) and higher (AB)ₙA amide oligomers where n≧2.

HPLC analysis of this product using the column and solvent system described for the Ethacure® 100 product did not result in clean separation of individual (AB)ₙA amide isomers (where n≧1) that are presumed to be present in this reaction product.

Proton NMR Spectroscopy. The 500 MHz proton NMR spectrum in DMSO of the product supported the desired structure of (AB)ₙA amide isomers (where n≧1) based on the presence of amide protons at 9.9-10.3 ppm, furanic protons at 7.36-7.45 ppm, phenyl ring proton between at 6.95-7.35 ppm, amine protons at 4.8-5.6 ppm, and methyl plus thiol-methyl protons at 1.90-2.50 ppm.

Infrared Spectroscopy. The infrared spectrum of this product showed a prominent peak at 1664 cm⁻¹ that is consistent with aromatic amides.

Titration. Titration of the crude reaction mixture dissolved in chlorobenzene with 0.1703N perchloric acid in glacial acetic acid using methyl violet as an indicator indicated that the amine content was 2.75 mmole amino groups per gram which is 74.6% of amino group concentration expected for an A-B-A system. The reduced amino group concentration is believed to be caused by the presence of higher (AB)ₙA systems where n≧2, in addition to A-B-A systems. Titration of Ethacure® 300 with this titrant system indicated that the amine concentration was 50.1% of theoretical and this result is assumed to result from the fact that once one amino group of Ethacure® 300 is protonated, the remaining amino group is insufficiently basic to become protonated. This reduced basicity of Ethacure® 300 relative to Ethacure® 100 could be due to the fact that the two methylthio groups of Ethacure® 300 are relatively electron withdrawing versus the ethyl groups of Ethacure® 100 that are electron donating.

### Example 9

This example illustrates the determination of the relative reactivities of an aromatic diamine (Ethacure® 100) and FDCA bisamide of an aromatic diamine (Ethacure® 100).

The relative reactivities of Ethacure® 100 and FDCA bisamide of Ethacure® 100 bisamide were determined by reacting various ratios of these materials with toluene diisocyanate (Tolonate®) in the presence of 1,4-butanediol and determining the viscosity of these mixtures with time using a rheometer (a Rheometric Scientific SR5® rheometer that was thermostatted to 25° C. and set to apply a constant stress of 20.0 dyne/cm² and a constant frequency of 1.0 rad/sec). Samples were mixed while adding Tolonate® last at essentially time zero on the viscosity/time plots. Three sets of concentrations were prepared with the following concentrations and the viscosity/time plots are shown in FIG. 4:

Composition 1 (square data points): 50.0 mg Ethacure® 100 (0.56 mmole amine groups), 290 mg 1,4-butanediol (3.2 mmole), 660 mg Tolonate® (3.2 mmole)-square data points in FIG. 4
Composition 2 (diamond data points): 26.5 mg Ethacure® 100 (0.30 mmole amine groups), 65.3 mg FDCA Ethacure® 100 bisamide (0.27 mmole amine groups), 290 mg 1,4-butanediol (3.2 mmole), 683 mg Tolonate® (3.3 mmole)-diamond data points in FIG. 4
Composition 3 (triangle data points): 138 mg FDCA Ethacure® 100 bisamide (0.0.56 mmole amine groups), 300 mg 1,4-butanediol (3.3 mmole), 690 mg Tolonate® (3.4 mmole)-triangular data points in FIG. 4

The total moles of amine groups in these three compositions were about the same while the mole ratios of amine groups supplied by Ethacure® 100 and FDCA bisamide of Ethacure® 100 were varied from 100:0 to approximately 50:50 to 0:100, while maintaining essentially the same concentrations of alcohol and isocyanate in these compositions. Thus, differences in the relative rates of viscosity increases were caused by differences in amine reactivities. The attained viscosities were a measure of the molecular weight attained, which is a measure of the degree of reaction of isocyanates functionality with amine and alcohol functionality. It can be seen in FIG. 4 that the relative rates of viscosity (plotted in Poise) increases were in the following order: Composition 1>Composition 2>Composition 3. Also, the relative ultimate viscosities attained were: Composition 1>Composition 2>Composition 3. Thus, the relative rates of these reactions were inversely related to the concentration of FDCA Ethacure® 100 bisamide amino group concentrations and it can be concluded that attachment of FDCA groups via amide linking functionality to one of the two amine groups of an Ethacure® 100 molecule, as is the case in FDCA Ethacure® 100 bisamide, significantly reduces the reactivity of the remaining amino group of Ethacure® 100 towards reaction with isocyanate functionality. Based on the principles involved, similar reductions in amino group reactivity would be expected in the (AB)ₙA polyamide compounds where n≧2 which are also terminated with amino groups. The same type reduction in relative reactivities of amine groups is also expected in the Ethacure® 300 when it is linked to FDCA via amide linkages to generate (AB)ₙA where n≧1 since the same principles will be in effect.

While the forms of the invention herein disclosed constitute presently preferred embodiments, many others are possible. It is not intended herein to mention all of the possible equivalent forms or ramifications of the invention. It is to be understood that the terms used herein are merely descriptive, rather than limiting, and that various changes may be made without departing from the spirit of the scope of the invention.

1. An aromatic amine bisamide of furan-2,5-dicarboxylic acid comprising:
a structure (AB)ₙA;
wherein A is an aromatic diamine moiety, B is a furan-2,5-dicarboxylic acid moiety and n is an integer from 1 to 10;
wherein each aromatic diamine moiety in the bisamide comprises 0, 1, 2, 3, 4, or 5 substituents selected from the group consisting of alkyl, aryl, alkylaryl, halogen, nitro, carboxyl, carbonyl, primary amino (—NH₂), secondary amino (—NHR), tertiary amino (—NR₂), aminoalkyl (—RNH₂), hydroxyl (—OH), alkoxy (—OR), hydroxylalkyl (—ROH), thiol (—SH), and alkylthio (—SR), wherein at least one group is either a primary or secondary amino, aminoalkyl, hydroxyl, or thiol group, and the remaining positions are occupied by H; and
wherein each group may contain between 1 to 10 carbon atoms.

2. The aromatic amine bisamide of furan-2,5-dicarboxylic acid according to claim 1, wherein the alkylthio group comprises the methylthio group.

3. The aromatic amine bisamide of furan-2,5-dicarboxylic acid according to claim 1,
wherein the group may contain up to 6 carbon atoms.

4. The aromatic amine bisamide of furan-2,5-dicarboxylic acid according to claim 1, comprising:
the specific positional labeling of the two nitrogen atoms in the major species A1 and A2 of the Ethacure® 100 series diamines as follows: 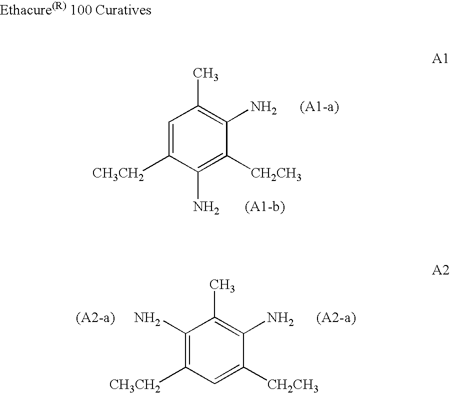
wherein the positional specificity of individual bisamides is specified by the following generically labeled structures where the label AX-y (where X=1 or 2 and y=a or b) specifies the specific aromatic nitrogen atom involved in amide bond formation:

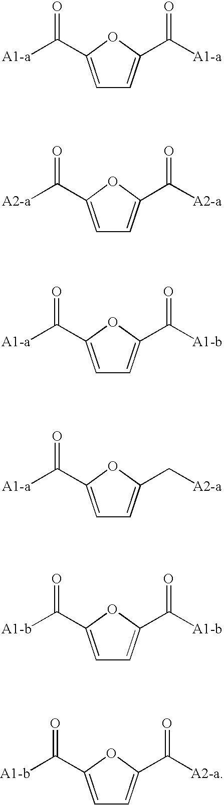

5. The aromatic amine bisamide of furan-2,5-dicarboxylic acid according to claim 1, comprising:
the specific positional labeling of the two nitrogen atoms in the major species A1 and A2 of the Ethacure® 300 series diamines as follows: 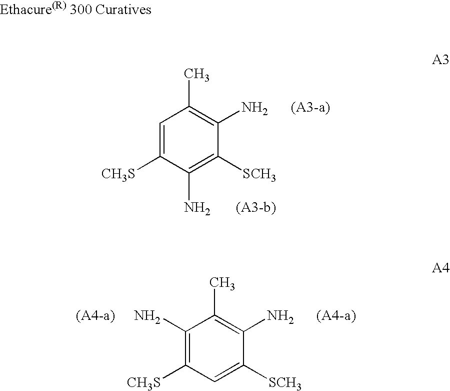
wherein the positional specificity of individual bisamides is specified by the following generically labeled structures where the label AX-y (where X=3 or 4 and y=a or b) specifies the specific aromatic nitrogen atom involved in amide bond formation:

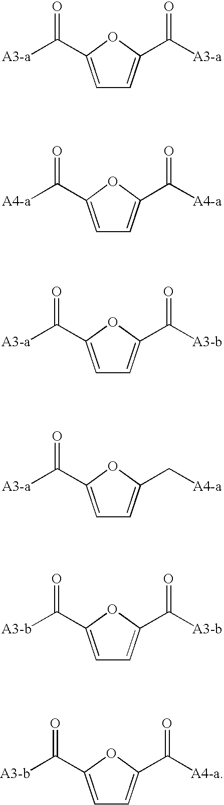

6. A further broad embodiment includes the composition comprising: 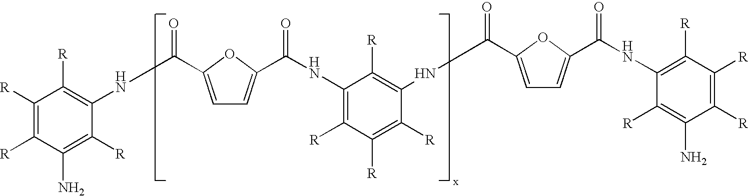
Wherein x=0, have A-B-A structure;
Wherein x=1 have A-B-A-B-A type structure;
Wherein x may have any value from 0 to 9;
the amino (—NH₂) groups on the substituted phenyl ring may be meta, ortho, or para with respect to each other,
R may be the same or different, and is selected from the group consisting of alkyl, aryl, alkylaryl, halogen, nitro, carboxyl, carbonyl, primary amino (—NH₂), secondary amino (—NHR′), tertiary amino (—NR₂′), aminoalkyl (—R′NH₂), hydroxyl (OH), alkoxy (—OR′), hydroxylalkyl (—R′OH), thiol (—SH) and alkylthio (—SR′), wherein the remaining positions are occupied by H, and
wherein the R an R′ groups may contain 1 to 10 carbon atoms.

7. A method for controlling cure time and (or) pot life of polyurea, hybrid epoxy-urethane, and hybrid urea-urethane chain extenders for polyurethane and polyurea elastomer systems comprising:
a. using an aromatic diamine curative, wherein the aromatic diamine is replaced to varying amounts with an furan-2,5-dicarboxylic acid bisamide of such aromatic diamine, wherein increasing amounts of furan-2,5-dicarboxylic acid bisamide lead to reduced reaction rates that provide increased pot life and longer reaction time.

8. A method for making furan-2,5-dicarboxylic acid bisamide comprising:
a. providing a furan-2,5-dicarboxylic acid diacid chloride, an aromatic diamine, an optional catalyst and a solvent;
b. mixing the furan-2,5-dicarboxylic acid diacid chloride with the aromatic diamine in the solvent, optionally in the presence of the catalyst; and
c. reacting the mixture of step b, optionally under heat, until the furan-2,5-dicarboxylic acid bisamide is formed.

9. The method according to claim 8, comprising separating the furan-2,5-dicarboxylic acid bisamide from the reaction mixture.

10. The method according to claim 9, wherein the furan-2,5-dicarboxylic acid bisamide is separated by filtration.

11. A method for separating a furan-2,5-dicarboxylic acid bisamide having the formula (A-B)ₙA wherein n=1, from higher oligomers having the formula (A-B)ₙA wherein n is greater or equal to 2, comprising:
obtaining a mixed (A-B)ₙ-A product, wherein n is 1 to greater than 1;
fractionating the mixed product with a moderately polar solvent in which the A-B-A is more soluble than the higher oligomers, wherein the A-B-A product is dissolved in the solvent.

12. The method according to claim 11, wherein the solvent is acetonitrile,

13. The method according to claim 11, wherein the solvent containing A-B-A product is removed by from the higher oligomers by filtration.

14. A method for making a furan-2,5-dicarboxylic acid bisamide comprising:
a. providing furan-2,5-dicarboxylic acid, aromatic diamine, triphenyl phosphite, and pyridine;
b. mixing furan-2,5-dicarboxylic acid, aromatic diamine, triphenyl phosphite, and pyridine; in solvent; and
c. reacting the mixture under optional heating until the furan-2,5-dicarboxylic acid bisamide is formed.

15. The method according to claim 8, comprising heating to a temperature of about 80° C. to about 110° C.

16. A method for making a furan-2,5-dicarboxylic acid bisamide comprising:
a. providing furan-2,5-dicarboxylic acid, aromatic diamine, a molecular sieve Zeolite® and an optional solvent;
b. mixing furan-2,5-dicarboxylic acid, aromatic diamine, molecular sieve Zeolite® and with or without the solvent;
triphenyl phosphite, and pyridine; in solvent; and
c. reacting the mixture with microwave radiation until the furan-2,5-dicarboxylic acid bisamide is formed.

17. A method for making a furan-2,5-dicarboxylic acid bisamide comprising:
a. providing furan-2,5-dicarboxylic acid, aromatic diamine, phosphorous pentachloride, and solvent;
b. mixing furan-2,5-dicarboxylic acid, aromatic diamine, phosphorous pentachloride, and solvent and heating; and
c. reacting the mixture until the furan-2,5-dicarboxylic acid bisamide is formed.
